(19) United States
(12) Patent Application Publication
(10) Pub. No.: US 2026/0148853 A1
(43) Pub. Date: May 28, 2026

(54) UQCC2-BASED COMPUTER-ASSISTED METHOD, SYSTEM, AND DEVICE FOR DIAGNOSING OSIMERTINIB-RESISTANT LUNG CANCER, PREDICTING DRUG THERAPEUTIC EFFECTS, AND SCREENING DRUGS TARGETED FOR OSIMERTINIB-RESISTANT LUNG CANCER

(71) Applicant: THE SECOND HOSPITAL OF DALIAN MEDICAL UNIVERSITY, Dalian (CN)

(72) Inventors: Xiaohui DU, Dalian (CN); Qi WANG, Dalian (CN); Han WANG, Dalian (CN); Hui ZHAO, Dalian (CN); Peipei HE, Dalian (CN); Ying LI, Dalian (CN); Congcong ZHANG, Dalian (CN)

(73) Assignee: THE SECOND HOSPITAL OF DALIAN MEDICAL UNIVERSITY, Dalian (CN)

(21) Appl. No.: 19/332,130

(22) Filed: Sep. 18, 2025

(30) Foreign Application Priority Data
Sep. 18, 2024 (CN) 202411300208.5

(51) Int. Cl. G16H 50/20; G16H 20/10

(52) U.S. Cl. CPC G16H 50/20; G16H 20/10

(57) ABSTRACT

UQCC2-based computer-assisted method, system, and device for diagnosing osimertinib-resistant lung cancer, predicting drug treatment effects, and screening drugs targeted for osimertinib-resistant lung cancer are provided. The method includes receiving input data, the input data including expression level data of UQCC2 in a patient with lung cancer; generating, based on the input data, an indication of whether the patient with lung cancer is a patient with osimertinib-resistant lung cancer through a machine learning model, a lung cancer including non-small cell lung cancer. The disclosure first discovers the significant overexpression of UQCC2 in the osimertinib-resistant lung cancer and the functional interaction between UQCC2 and METTL3, and provides an efficient and rapid method for diagnosing the patient with osimertinib-resistant lung cancer, predicting the drug treatment effect on the patient with osimertinib-resistant lung cancer, and screening drugs based on targeting UQCC2 and METTL3, demonstrating critical research significance for related disease prevention and treatment.

### CROSS-REFERENCE TO RELATED DISCLOSURES

This application claims priority to Chinese Patent Application No. 202411300208.5, filed on Sep. 18, 2024, the entire contents of which are hereby incorporated by reference.

### TECHNICAL FIELD

This disclosure relates to the field of intelligent medicine, and in particular, to a ubiquinol-cytochrome c reductase complex assembly factor 2 (UQCC2)-based computer-assisted method, system, and device for diagnosing patients with osimertinib-resistant lung cancer, predicting drug treatment effects, and screening drugs.

### BACKGROUND

Non-small cell lung cancer (NSCLC), the primary subtype of lung cancer, is one of the most common malignant tumors, with an extremely high incidence and mortality worldwide. In patients with NSCLC, genetic mutations and alterations, especially the L858R and T790M mutations in epidermal growth factor receptor (EGFR), are the main pathogenic causes. Therefore, targeted therapy with EGFR tyrosine kinase inhibitors (TKIs), such as gefitinib and erlotinib, has become standard first-line therapy for the treatment of patients with NSCLC with EGFR mutations. Osimertinib (AZD9291), a third-generation EGFR-TKI for the treatment of NSCLC patients with gefitinib or erlotinib resistance, has made significant progress. However, osimertinib resistance inevitably develops, leading to treatment failures and deaths of 36 patients (A. Noronha, et al. Cancer Discov 2022, 12 (11), 2666, https://doi.org/10.1158/2159-8290.Cd-22-0111; J. C. Madukwe, Cell 2023, 186 (8), 1515, https://doi.org/10.1016/j. cell.2023.03.019.). Hence, elucidating the underlying mechanisms of osimertinib resistance and discovering novel medications to overcome drug resistance are urgently required.

### SUMMARY

In view of this, the purpose of the present disclosure is to provide a UQCC2-based computer-assisted method, system, and device for diagnosing a patient with osimertinib-resistant lung cancer, predicting a drug treatment effect, and screening drugs.

In order to achieve the above object, one or more embodiments of the present disclosure provide a computer-assisted method for diagnosing a patient with osimertinib-resistant lung cancer. The computer-assisted method may include receiving input data, the input data including expression level data of UQCC2 in a patient with lung cancer; and generating, based on the input data, an indication of whether the patient with lung cancer is the patient with osimertinib-resistant lung cancer through a machine learning model, where the lung cancer includes a non-small cell lung cancer.

One or more embodiments of the present disclosure provide a computer-assisted system for diagnosing a patient with osimertinib-resistant lung cancer. The computer-assisted system may include a data acquisition unit, a data evaluation unit, and a drug-resistant identification unit. The data acquisition unit may be configured to obtain expression level data of UQCC2 in the patient with lung cancer. The data evaluation unit may include stored reference data and a data processor, and the data processor may include an algorithm for comparing the expression level data of UQCC2 with the stored reference data. The drug-resistant identification unit may be configured to determine whether the patient with lung cancer is the patient with osimertinib-resistant lung cancer based on the algorithm for comparing the expression level data of UQCC2 in the patient with lung cancer with the stored reference data, and the lung cancer may include a non-small cell lung cancer.

One or more embodiments of the present disclosure provide a computer-assisted device for diagnosing a patient with osimertinib-resistant lung cancer. The computer-assisted device may include a storage and a processor, the storage may be configured to store program instructions, and the processor may be configured to invoke the program instructions and realize the computer-assisted method for diagnosing the patient with osimertinib-resistant lung cancer when the program instructions are executed.

One or more embodiments of the present disclosure provide a non-transitory computer-readable storage medium storing a computer program. The computer-assisted method for diagnosing the patient with osimertinib-resistant lung cancer may be implemented when a processor executes the computer program.

One or more embodiments of the present disclosure provide a computer-assisted method for predicting a drug treatment effect on a patient with osimertinib-resistant lung cancer. The computer-assisted method may include receiving input data, the input data including expression level data of UQCC2 in the patient with osimertinib-resistant lung cancer after treatment with a drug; and generating, based on the input data, an indication of whether the drug has a treatment effect on the patient with osimertinib-resistant lung cancer through a machine learning model, where the drug includes osimertinib, and the lung cancer includes a non-small cell lung cancer.

One or more embodiments of the present disclosure provide a computer-assisted device for predicting a drug treatment effect on a patient with osimertinib-resistant lung cancer. The computer-assisted device may include a storage and a processor. The storage may be configured to store program instructions, and the processor may be configured to invoke the program instructions and realize the method for predicting the drug treatment effect on the patient with osimertinib-resistant lung cancer when the program instructions are executed.

One or more embodiments of the present disclosure provide a non-transitory computer-readable storage medium storing a computer program. The computer-assisted method for predicting the drug treatment effect on the patient with osimertinib-resistant lung cancer may be implemented when a processor executes the computer program.

### BRIEF DESCRIPTION OF THE DRAWINGS

The present disclosure is further illustrated in terms of exemplary embodiments. These exemplary embodiments are described in detail with reference to the drawings. These embodiments are non-limiting exemplary embodiments, in which like reference numerals represent similar structures, and wherein:

FIG. 1 is a flowchart illustrating an exemplary computer-assisted method for diagnosing a patient with osimertinib-resistant lung cancer according to some embodiments of the present disclosure;

FIG. 2 is a schematic diagram illustrating an exemplary computer-assisted system for diagnosing a patient with osimertinib-resistant lung cancer according to some embodiments of the present disclosure;

FIG. 3 is a schematic diagram illustrating an exemplary computer-assisted device for diagnosing a patient with osimertinib-resistant lung cancer according to some embodiments of the present disclosure;

FIG. 4 is a flowchart illustrating an exemplary computer-assisted method for predicting a drug treatment effect on a patient with osimertinib-resistant lung cancer according to some embodiments of the present disclosure;

FIG. 5 is a schematic diagram illustrating an exemplary computer-assisted system for predicting a drug treatment effect on a patient with osimertinib-resistant lung cancer according to some embodiments of the present disclosure;

FIG. 6 is a flowchart illustrating an exemplary UQCC2 and methyltransferase 3, N6-adenosine-methyltransferase complex catalytic subunit(METTL3)-based computer-assisted method for screening drugs according to some embodiments of the present disclosure;

FIG. 7 is a schematic diagram illustrating an exemplary UQCC2 and METTL3-based computer-assisted system for screening drugs according to some embodiments of the present disclosure;

FIG. 8 is a graph illustrating results of gene expressions of NCI-H1975 cells and osimertinib-resistant cells (Osim) treated with osimertinib, as detected and analyzed by methylated RNA immunoprecipitation sequencing (MeRIP-seq) and RNA sequencing (RNA-seq);

FIG. 9 is a graph illustrating protein expression levels and corresponding statistical analysis of UQCC2 in NCI-H1975 cells and NCI-H1975/OR cells (treated with Ctrl, Osim, or Lomi alone or in combination) as determined by western blot assay, where NCI-H1975/OR cells (Ctrl) are osimertinib-resistant cells, NCI-H1975/OR cells (Osim) are osimertinib-resistant cells treated with osimertinib, NCI-H1975/OR cells (Lomi) are osimertinib-resistant cells treated with lomitapide, and NCI-H1975/OR cells (comb) are osimertinib-resistant cells treated with a combination of osimertinib and lomitapide;

FIG. 10 is a graph illustrating alteration results of N⁶-methyladenosine (m⁶A) abundance of UQCC2 in non-small cell lung cancer parental cells (H1975) and in osimertinib-resistant cells (Osim) treated with osimertinib through Integrative Genomics Viewer (IGV) analysis;

FIG. 11 is a graph illustrating expression levels of various biomarkers determined by western blot assay, where FIG. 11A is a graph illustrating protein expression levels and corresponding statistical analysis of METTL3 in NCI-H1975 cells and NCI-H1975/OR cells (treated with Ctrl, Osim, or Lomi alone or in combination) as determined by western blot assay; FIG. 11B is a graph illustrating an effect and corresponding statistical analysis of METTL3 knockdown on protein expression levels of UQCC2, P62, and LC3; and FIG. 11C is a graph illustrating an effect and corresponding statistical analysis of UQCC2 knockdown on protein expression levels of METTL3, P62, and LC3; and

FIG. 12 is a graph illustrating an effect of UQCC2 knockdown on an expression of LC3 determined by an immunofluorescence assay.

### DETAILED DESCRIPTION

In order to enable those in the art to better understand the embodiments of the present disclosure, the technical solutions in the embodiments of the present disclosure will be clearly and completely described below in conjunction with the accompanying drawings in the embodiments of the present disclosure.

In some of the processes described in the present disclosure and claims and in the above-described accompanying drawings, a plurality of operations appearing in a particular order are included, but it should be clearly understood that these operations may be performed in a different order than the order in which they appear herein, or performed in parallel. Serial numbers of the operations, such as S101, S102, etc., are merely used to distinguish between the various operations, and the serial numbers themselves do not represent any order of execution. Alternatively, the processes may include more or fewer operations, and the operations may be executed sequentially or in parallel.

The technical solutions in the embodiments of the present disclosure will be clearly and completely described below in conjunction with the accompanying drawings in the embodiments of the present disclosure. Obviously, the described embodiments are only a portion of the embodiments of the present disclosure and not all of the embodiments. Based on the embodiments in the present disclosure, all other embodiments obtained by a person skilled in the art without making creative labor fall within the scope of protection of the present disclosure.

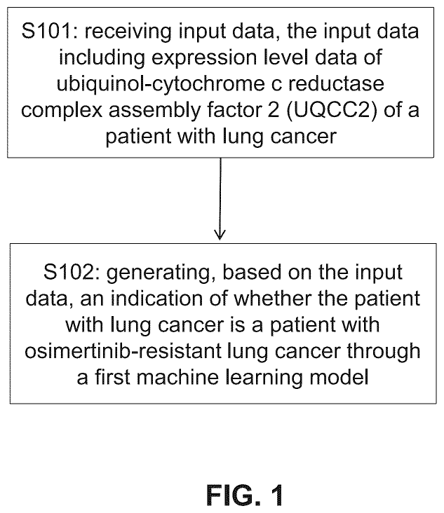

FIG. 1 is a flowchart illustrating an exemplary computer-assisted method for diagnosing a patient with osimertinib-resistant lung cancer according to some embodiments of the present disclosure. The computer-assisted method is performed by a processor, including the following operations.

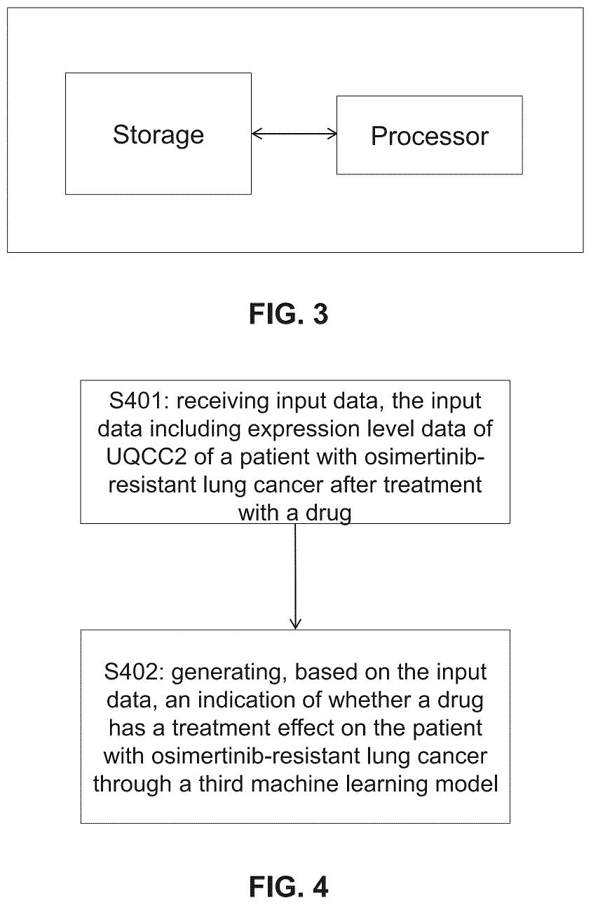

More descriptions of the processor may be found in FIG. 3 and related descriptions thereof.

The computer-assisted method for diagnosing the patient with osimertinib-resistant lung cancer may be performed by inputting clinical information, etc., of the patient into a computer, and outputting, from the computer, whether the patient with lung cancer is the patient with osimertinib-resistant lung cancer. The computer may include a desktop computer or a server, etc.

In S101, input data is received, and the input data includes expression level data of UQCC2 in the patient with lung cancer.

In some embodiments, the input data in S101 may also be referred to as first input data. The first input data refers to data related to the patient with lung cancer. The patient with lung cancer includes a patient with osimertinib-resistant lung cancer and a patient with osimertinib-non-resistant lung cancer. An operator (e.g., a physician, etc.) inputs data such as the measured expression level data of UQCC2 in the patient with lung cancer into the computer, and receives output information indicating whether the patient with lung cancer is the patient with osimertinib-resistant lung cancer from the computer.

In some embodiments, the expression level data of UQCC2 may be data that reflects the expression of UQCC2. The expression level data of UQCC2 includes a protein expression level, a nucleic acid expression level, etc., of UQCC2.

In one embodiment, a manner for measuring the protein expression level of UQCC2 includes, but is not limited to, western blot, immunohistochemistry, immunofluorescence, an enzyme-linked immunosorbent assay, mass spectrometry, flow cytometry, etc.

In one embodiment, a manner for measuring the nucleic acid expression level of UQCC2 includes, but is not limited to, a reverse transcription polymerase chain reaction (RT-PCR) manner, a quantitative reverse transcription polymerase chain reaction (qRT-PCR) manner, a biochip assay, Southern blotting, Northern blotting, gene microarray, etc.

In one embodiment, the lung cancer includes at least one of a small cell lung cancer, a non-small cell lung cancer, or the like.

In one embodiment, the non-small cell lung cancer includes at least one of squamous cell carcinoma, adenocarcinoma, large cell carcinoma, etc.

In one embodiment, the collection sample of UQCC2 of the patient with lung cancer may be from at least one of blood, tissue, feces, urine, etc.

In S102, based on the input data, an indication of whether the patient with lung cancer is the patient with osimertinib-resistant lung cancer is generated through a machine learning model.

The patient with osimertinib-resistant lung cancer is a patient with lung cancer who is resistant to osimertinib.

In some embodiments, the machine learning model in S102 is also referred to as a first machine learning model. In some embodiments, the first machine learning model includes, but is not limited to, at least one of a support vector machine learning model, a linear discriminant analysis model, a recursive feature removal model, a predictive analytics for microarray model, a logistic regression model, a CART algorithm, a flextree algorithm, a LART algorithm, a random forest algorithm, a MART algorithm, a machine learning algorithm, a penalized regression manner, etc., or any combinations thereof.

In some embodiments, the construction process of the first machine learning model includes the following operations.

First, the expression level data of UQCC2 in the patient with osimertinib-non-resistant lung cancer and the patient with osimertinib-resistant lung cancer are obtained from historical data.

Second, the acquired expression level data is divided randomly into training sets and test sets, and block processing is performed on the training sets and the test sets, etc. Both the training sets and the test sets include the expression level data of UQCC2 in the patient with osimertinib-non-resistant lung cancer and the patient with osimertinib-resistant lung cancer.

Third, parameter adjusting and training are performed on the first machine learning model corresponding to the patient with lung cancer by using the expression level data of UQCC2 in the training sets to generate the first machine learning model. Intensive training is performed on the first machine learning model by using the expression level data of UQCC2 in the test sets to obtain the trained first machine learning model.

The first machine learning model includes a diagnostic threshold of the expression level data of UQCC2. The first machine learning model may compare the magnitudes of the expression level data and the diagnostic threshold. When the expression level data of UQCC2 is higher than the diagnostic threshold, the first machine learning model determines that the patient with lung cancer is the patient with osimertinib-resistant lung cancer. When the expression level data of UQCC2 is not higher than the diagnostic threshold, the first machine learning model determines that the patient with lung cancer is the patient with osimertinib-non-resistant lung cancer.

In some embodiments, the diagnostic threshold includes, but is not limited to, thresholds corresponding to performance indicators such as an accuracy (Acc), an area under the curve (AUC), a sensitivity, a true negative rate (TNR), a positive predictive value (PPV), a negative predictive value (NPV), etc. The diagnostic threshold may be other performance indicators defined according to a specific application scenario, a composite performance indicator and its corresponding threshold designed according to a specific preset algorithm on the basis of each of the above performance indicators, etc., which is not limited in the present disclosure.

In some embodiments, when there is a plurality of first machine learning models corresponding to each of the performance indicators, the optimal first machine learning model may be selected based on the performance indicators prioritized in different application scenarios. As a result, through the testing of the test sets, it may be ensured that the trained first machine learning model has a strong robustness and generalization ability, which is not only applicable to the samples in the training sets, but also applicable to new samples that have not participated in the training.

In some embodiments, the processor may detect and analyze the gene expressions (e.g., the protein expression level, etc.) of non-small cell lung cancer cell lines (e.g., NCI-H1975 cells (Control, Ctrl) and NCI-H1975/OR cells (Osimertinib, Osim)) by MeRIP-seq and RNA-seq, etc. NCI-H1975/OR cells (Osim) are osimertinib-resistant cells.

The specific operations include, after extracting total RNA from the NCI-H1975 cells and the NCI-H1975/OR cells (Osim), isolating the total RNA by using an RNA isolation manner (e.g., TRIzol (Invitrogen), etc.), quantifying the total RNA by using UV spectrophotometer (NanoDrop), etc., and performing integrity testing on RNA by using Bioanalyzer (Agilent), etc. When the concentration of RNA is greater than 50 ng/μL, an RNA integrity score (e.g., RIN value, etc.) is greater than 7.0, an RNA purity (e.g., OD260/280, etc.) is greater than 1.8, and an extraction amount of the total RNA (denoted as total RNA) is greater than 50μg, then downstream experiments are met. The specific operations also include performing purification on the total RNA to obtain Poly(A) RNA by using a specific purification manner (e.g., Dynabeads Oligo (dT), etc.), and fragmenting the captured Poly(A) RNA by using a Magnesium Ion Interrupter Kit under high-temperature (e.g., 94° C., etc.) conditions; isolating the portion of RNA in the Poly(A) RNA fragment that binds to the m⁶A-specific antibody (Synaptic Systems); performing reverse transcription on the portion of RNA (Immunoprecipitated RNA, IP RNA) that binds to the m⁶A-specific antibody (Synaptic Systems) by a reverse transcription manner (e.g., SuperScript II, etc.), then synthesizing U-labeled second-strand DNA, and amplifying a ligated product (e.g., the U-labeled second-strand DNA) by a PCR amplification manner, etc., to obtain an IP RNA library; performing paired-end sequencing (e.g., PE150, etc.) on the total RNA (input) and the IP RNA library, etc., using an Illumina NovaSeq 6000platform (LC-Bio Technology Co, Ltd, China), etc. The total RNA (input) may be an RNA library obtained by reverse transcription and amplification using the portion of RNA that binds to m⁶A-specific antibody (Synaptic Systems) that is not isolated. The IP RNA library may be an RNA library obtained by reverse transcription and amplification of IP RNA.

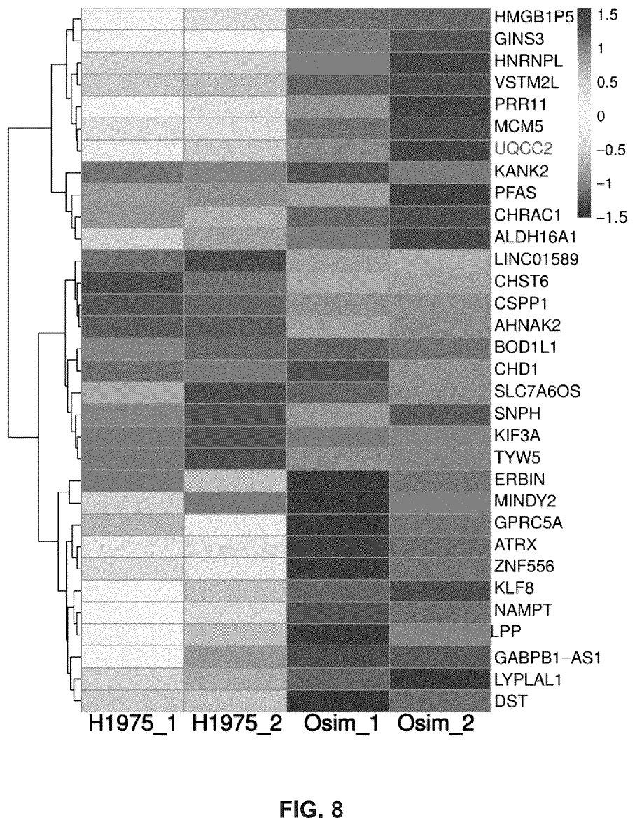

FIG. 8 is a graph illustrating results of gene expressions of NCI-H1975 cells and osimertinib-resistant cells (Osim) treated with osimertinib, as detected and analyzed by MeRIP-seq and RNA-seq.

As shown in FIG. 8, in the rows of UQCC2 highlighted in red, boxes corresponding to NCI-H 1975/OR cells (Osim) are red (indicating a value greater than 0), and boxes corresponding to NCI-H1975 cells (Ctrl) are blue (indicating a value less than 0), indicating that the expression level of UQCC 2 in the NCI-H 1975/OR cells (Osim) is higher than the expression level of UQCC2 in the NCI-H1975 cells (Ctrl).

In a specific embodiment, the protein expression levels of UQCC2 in the NCI-H1975 cells and the NCI-H1975/OR cells (Ctrl) were determined by western blot assay, or the like. The NCI-H1975/OR cells (Ctrl) were osimertinib-resistant cells. The western blot assay used anti-UQCC2 (antibody dilution ratio of 1:1000, Proteintech, #25781-1-AP). Anti-GAPDH (antibody dilution ratio of 1:50,000, Proteintech, #60004-1-Ig) was used as a loading control. Significance (denoted as P) was determined using GraphPad Prism software, etc. All statistical analyses were performed using data from at least three independent experiments. More than three means were analyzed using manners such as two-way ANOVA or one-way ANOVA followed by Bonferroni correction, and two means were analyzed using unpaired Student's t-tests for comparison. *P□0.05 indicates that the difference is statistically significant. “*” refers to a marker sign of significance.

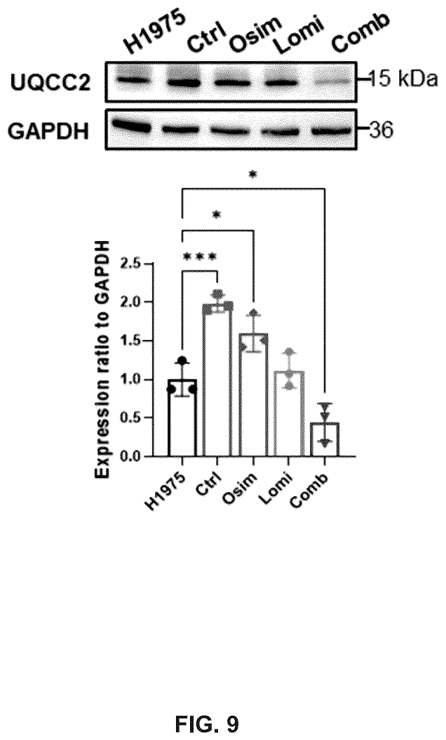

FIG. 9 is a graph illustrating protein expression levels and corresponding statistical analysis of UQCC2 in NCI-H1975 cells and NCI-H1975/OR cells (treated with Ctrl, Osim, or Lomi alone or in combination) as determined by western blot assay, where NCI-H1975/OR cells (Ctrl) are osimertinib-resistant cells, NCI-H1975/OR cells (Osim) are osimertinib-resistant cells treated with osimertinib, and NCI-H1975/OR cells (Lomi) are osimertinib-resistant cells treated with lomitapide.

As shown in FIG. 9, the column corresponding to the NCI-H1975/OR cells (Ctrl) is higher than the column corresponding to the NCI-H1975 cells, demonstrating that the expression level of UQCC2 in the NCI-H1975/OR cells (Ctrl) is significantly higher than the expression level of UQCC2 in the NCI-H1975 cells. In FIG. 9, “*” indicates p□0.05, “**” indicates p□0.01, “***” indicates p□0.001, “****” indicates p□0.0001, and ns indicates non-significance.

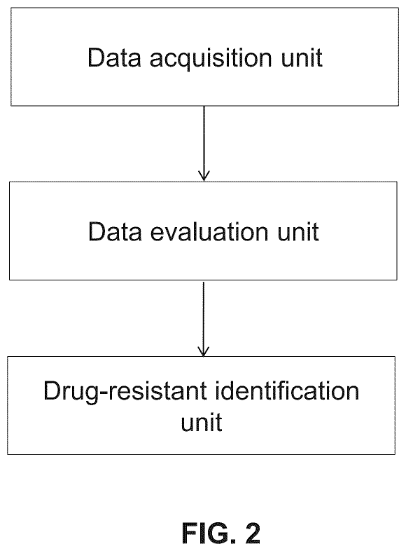

FIG. 2 is a schematic diagram illustrating an exemplary computer-assisted system for diagnosing a patient with osimertinib-resistant lung cancer according to some embodiments of the present disclosure. The computer-assisted system includes a data acquisition unit, a data evaluation unit, and a drug-resistant identification unit.

The data acquisition unit is configured to obtain the expression level data of UQCC2 in the patient with lung cancer.

The data evaluation unit includes stored reference data and a data processor, and the data processor includes an algorithm for comparing the expression level data of UQCC2 with the stored reference data. The data processor is configured to execute algorithms or instructions, etc., including the processor, etc., shown in FIG. 3.

The reference data may be preset, which is the expression level data of UQCC2 used for reference.

The drug-resistant identification unit is configured to determine whether the patient with lung cancer with osimertinib-resistant lung cancer, based on the algorithm for comparing the expression level data of UQCC2 in the patient with lung cancer with the stored reference data. The lung cancer includes the non-small cell lung cancer, etc.

FIG. 3 is a schematic diagram illustrating an exemplary computer-assisted device for diagnosing a patient with osimertinib-resistant lung cancer according to some embodiments of the present disclosure. The computer-assisted device includes a storage and a processor.

The storage is configured to store program instructions. The processor is configured to invoke the program instructions and realize the above computer-assisted method for diagnosing the patient with osimertinib-resistant lung cancer when the program instructions are executed.

In some embodiments, the processor may include a central processing unit (CPU), an application-specific integrated circuit (ASIC), an application-specific instruction set processor (ASIP), a graphics processing unit (GPU), a physics processing unit (PPU), etc., or any combination thereof. The storage may include a mass storage, etc., or any combination thereof.

In some embodiments, the storage may be integrated in the processor. The data acquisition unit, the data evaluation unit, and the drug-resistant identification unit may also be integrated into the processor.

Embodiments of the present disclosure also provide a non-transitory computer-readable storage medium storing a computer program. The aforementioned computer-assisted method for diagnosing the patient with osimertinib-resistant lung cancer is implemented when the computer program is executed by a processor.

In some embodiments, when the patient with lung cancer is the patient with osimertinib-resistant lung cancer, a target drug is administered to the patient with lung cancer, and the target drug may include lomitapide.

In some embodiments, when a patient with lung cancer is diagnosed with osimertinib-resistant lung cancer, a candidate drug is administered to the patient with lung cancer. The candidate drug is obtained as follows. The processor may determine a plurality of first candidate molecules and synthetic feasibilities of the plurality of first candidate molecules based on protein structural information, a chemical valence bonding rule, and a pharmacophore module through a drug generation model. The processor may determine a second candidate molecule from the plurality of first candidate molecules based on the synthetic feasibilities and obtain the candidate drug based on the second candidate molecule.

The candidate drug refers to a drug intended for treating the patient with osimertinib-resistant lung cancer.

In some embodiments, the process of verifying the candidate drug is also included, such as verifying that the candidate drug may inhibit the proliferation of osimertinib-resistant lung cancer cell lines using cell experiments, or verifying that the candidate drug may inhibit tumor growth in animals with osimertinib-resistant lung cancer tumors or improve the survival rate of such animals using animal experiments. The protein structural information refers to information related to the spatial structure of a binding site of a UQCC2 protein to a METTL3 protein. In some embodiments, the protein structural information includes a three-dimensional conformation of the binding site, an arrangement of atoms of the binding pocket, and a space charge distribution. More descriptions regarding the binding site may be found in the descriptions below and the related sections.

In some embodiments, the protein structural information may be obtained from the storage or a third-party database (e.g., the Protein Data Bank, etc.).

The chemical valence bonding rule refers to a set of chemical rules that limit the way atoms bond in molecules, e.g., the maximum connectivity for each atom type and the specifications for ring structure construction.

In some embodiments, the chemical valence bonding rules may be obtained from the storage.

The pharmacophore module refers to a structural fragment with biologically active effects that is extracted from existing small molecule drugs. In some embodiments, the pharmacophore module includes an electron-donating/withdrawing region, a hydrophobic group, an aromatic ring, etc. More descriptions regarding the pharmacophore module may be found in the related descriptions below and the related sections.

In some embodiments, the pharmacophore module may be obtained from the storage.

In some embodiments, the drug generation model may be a machine learning model (which may also be referred to as a second machine learning model). For example, the drug generation model may include any one or a combination of, for example, a generative adversarial network (GAN) model, other customized model structures, etc.

In some embodiments, the drug generation model includes a molecular generation model and an evaluation model.

In some embodiments, inputs of the molecular generation model may be the protein structural information, the pharmacophore module, and the chemical valence bonding rule, and outputs of the molecular generation model may be the plurality of first candidate molecules.

The first candidate molecules refer to drug molecules to be determined for treating the patient with osimertinib-resistant lung cancer.

In some embodiments, inputs of the evaluation model may include the first candidate molecules, and outputs of the evaluation model may be the synthetic feasibilities corresponding to the first candidate molecules.

The synthesis feasibilities are used to characterize the likelihood that the first candidate molecules may be synthesized. In some embodiments, the synthesis feasibilities may be represented by numerical values, e.g., a value of 1 indicates that the first candidate molecules are synthesizable, and a value of 0 indicates that the first candidate molecules are not synthesizable.

In some embodiments, the molecular generation model and the evaluation model may be obtained by joint training of a plurality of sets of training samples with labels. The training samples may include sample protein structural information, a sample pharmacophore module, and a sample chemical valence bonding rule, and labels may be actual synthetic feasibilities of drug molecules corresponding to the training samples.

In some embodiments, the processor may obtain the training samples and the labels from historical data. The actual synthesis feasibilities of the drug molecules may be determined by the technician labeling.

In some embodiments, the evaluation model and the molecular generation model may also be obtained by joint training. The processor may input the plurality of training samples into an initial molecular generation model to obtain a plurality of sample drug molecules output from the initial molecular generation model, and input the plurality of sample drug molecules into the initial evaluation model, construct a loss function from the labels and an output result of the initial evaluation model, and iteratively update parameters of the initial molecular generation model and the initial evaluation model based on the loss function by a gradient descent algorithm, etc. When preset training conditions are satisfied, the trained molecular generation model and evaluation model are obtained. The preset training conditions may be that the loss function converges, the count of iterations reaches a threshold, or the like.

In some embodiments, the drug generation model may also be obtained by a plurality of rounds of iterations. One iteration among the plurality of rounds of iterations includes inputting the training samples and random noise into the initial molecular generation model to obtain a type I molecule and setting a first label for the type I molecule. The training samples include sample protein structural information, a sample chemical valence bonding rule, and a sample pharmacophore module. The iteration also include obtaining a type II molecule from the molecular library and setting a second label for the type II molecule, updating parameters of the initial evaluation model based on the type I molecule, the type II molecule, the first label, and the second label, keeping parameters of an updated initial evaluation model, changing the first label of the type I molecule to a third label and inputting the third label into the updated initial evaluation model, and updating, based on the third label and an output result of the updated initial evaluation model, the parameters of the initial molecular generation model.

The random noise refers to interfering data that is randomly generated, e.g., an irregular drug molecule, other molecules not related to the drug molecules, etc.

The type I molecule refers to a drug molecule output by the initial molecular generation model. The first label is a label of the type I molecule. In some embodiments, the first label may be labeled as a “false source” and is indicated by the label value of 0.

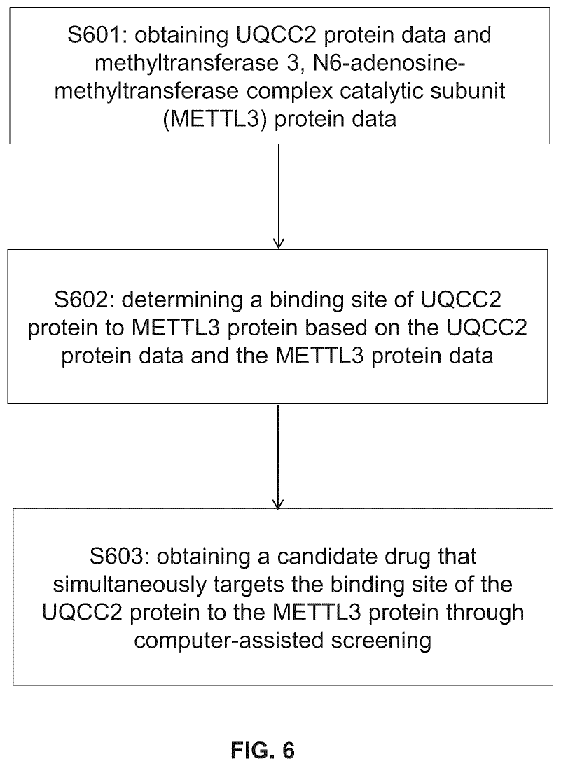

The type II molecule refers to an authentic drug molecule that corresponds to the sample protein structural information. The second label is a label of the type II molecule. In some embodiments, the second label may be labeled as “authentic source” and is indicated by the label value of 1. The processor may obtain the type II molecule whose protein structural information is the same as the sample protein structural information from the molecular library. More descriptions regarding the molecular library may be found in FIG. 6 and related descriptions thereof.

It is understood that the type I molecule is a virtual drug molecule output from the initial molecular generation model, and the type II molecule is an authentic drug molecule.

In some embodiments, the processor may initialize the molecular generation model and the evaluation model to obtain the initial molecular generation model and the initial evaluation model and input the type I molecule and the type II molecule into the initial evaluation model. The processor may adopt a binary classification loss function, designate the first label and the second label as training targets, and update the parameters of the initial evaluation model through a backpropagation algorithm, etc., to improve the ability of the initial evaluation model to discriminate the synthesis feasibilities.

In some embodiments, the processor may keep the parameters of the updated initial evaluation model, change the first label of the type I molecule to a third label and input the third label into the updated initial evaluation model, calculate a binary classification loss between the third label and the output result of the updated initial evaluation model, and update the parameters (e.g., automatically removing a part of the random noise or mapping the random noise into synthesizable molecular structures as much as possible) of the initial molecular generation model exclusively by the backpropagation algorithm, so that the type I molecule generated by the initial molecular generation model is more likely to be determined as the “authentic source” by the initial evaluation model. The third label may be labeled as the “authentic source” and indicated by the label value of 1.

In some embodiments, in response to determining that the updated initial evaluation model and the updated initial molecular generation model do not satisfy a preset convergence condition, the updated initial evaluation model and the updated initial molecular generation model are designated as an initial evaluation model and an initial molecular generation model to be trained in the next round of iteration. In response to determining that the updated initial evaluation model and the updated initial molecular generation model satisfy the preset convergence condition, the updated initial evaluation model and the updated initial molecular generation model are designated as the evaluation model and the molecular generation model. The preset convergence condition may include the count of iterations reaching a threshold, etc.

According to some embodiments of the present disclosure, by alternately optimizing the parameters of the initial evaluation model and the initial molecular generation model, the drug generation model may continuously improve the biological activity of the first candidate molecules screened by the drug generation model, as well as the accuracy of the drug generation model in determining the synthetic feasibilities of the first candidate molecules. In the iteration, the alteration of parameter fixing and parameter updating may prevent the drug generation model from falling into pattern collapse or over-fitting, which ensures the diversity and practicality of the structures of the first candidate molecules.

The second candidate molecule refers to the determined drug molecule intended for the treatment of the patient with osimertinib-resistant lung cancer.

In some embodiments, the processor may determine the second candidate molecule from the plurality of first candidate molecules based on the synthesis feasibilities. For example, the processor may determine a first candidate molecule with a synthesis feasibility of 1 as the second candidate molecule based on the synthesis feasibilities of the plurality of first candidate molecules.

In some embodiments, the processor may obtain the candidate drug based on the second candidate molecule. For example, the processor obtains, based on the second candidate molecule, the candidate drug through molecular docking or pharmacophore mapping. More descriptions regarding the molecular docking and the pharmacophore mapping may be found in the descriptions below and the related sections.

According to some embodiments of the present disclosure, through obtaining the first candidate molecules by the drug generation model, the first candidate molecules with novel structures and precise target points may be generated in a structural space of an unknown compound, and practical synthesis feasibilities of the first candidate molecules are automatically assessed, which significantly broaden the exploration scope of the candidate drug.

In some embodiments, the processor may also obtain a similar molecule by searching in a molecular library based on the second candidate molecule, and determine the candidate drug based on the similar molecule.

The molecular library refers to an existing small molecule database, e.g., ZINC, ChEMBL, PubChem, etc., and the molecular library contains a plurality of known chemical structures and pharmacological activities of the chemical structures. The molecular library may provide diverse samples of molecular structures that may support drug screening. More descriptions regarding the molecular library may be found in the description below and the related sections.

The similar molecule refers to a drug molecule that is structurally similar to the second candidate molecule.

In some embodiments, the processor may search the molecular library based on the molecular structure of the second candidate molecule, and determine the drug molecule in the molecular library with a similarity that exceeds a similarity threshold as the similar molecule. The similarity threshold may be set empirically.

In some embodiments, the processor may obtain the candidate drug by further screening retrieved similar molecules.

In some embodiments, a manner for screening includes molecular docking, pharmacophore mapping, etc. More descriptions regarding the molecular docking and the pharmacophore mapping may be found in FIG. 6 and related descriptions thereof.

According to some embodiments of the present disclosure, after obtaining the second candidate molecule using the drug generation model, the processor further compares the chemical information in the existing molecular library with the second candidate molecule to quickly screen for a candidate drug with a molecular structure similar to that of the second candidate molecule, which not only improves the efficiency of drug screening, but also expands the candidate range of potential drugs, accelerating the drug discovery process and increasing the success rate of drug screening.

FIG. 4 is a flowchart illustrating an exemplary computer-assisted method for predicting a drug treatment effect on a patient with osimertinib-resistant lung cancer according to some embodiments of the present disclosure. The computer-assisted method includes the following operations.

In S401, input data is received, and the input data includes expression level data of UQCC2 in the patient with osimertinib-resistant lung cancer after treatment with a drug.

In some embodiments, the input data in S401 may also be referred to as second input data. The second input data refers to data related to the patient with osimertinib-resistant lung cancer. An operator (e.g., a physician) inputs the measured expression level data of UQCC2 in the patient with osimertinib-resistant lung cancer into the computer and receives information of whether the drug has treatment effects on the patient with osimertinib-resistant lung cancer output from the computer.

The expression level data of UQCC2 after treatment with a drug may be data that can reflect the expression of UQCC2 in the patient with osimertinib-resistant lung cancer after treatment with a drug.

In some embodiments, the expression level data of UQCC2 includes a protein expression level and a nucleic acid expression level of UQCC2.

More descriptions regarding the manner for obtaining the expression level data of UQCC2 may be found in FIG. 1 and related descriptions thereof.

In S402, based on the input data, an indication of whether the drug has a treatment effect on the patient with osimertinib-resistant lung cancer is generated through a machine learning model (also referred to as a third machine learning model).

In some embodiments, the drug includes, for example, osimertinib, lomitapide, etc.

In some embodiments, the third machine learning model includes, but is not limited to, at least one of a support vector-based machine learning model, a linear discriminant analysis model, a recursive feature elimination model, a microarray prediction analysis model, a logistic regression model, a CART algorithm, a flextree algorithm, a LART algorithm, a random forest algorithm, a MART algorithm, a machine learning algorithm, a penalized regression manner, etc., and any combination thereof.

In some embodiments, the manner for constructing the third machine learning model includes the following operations.

The expression level data of UQCC2 in the patient with osimertinib-resistant lung cancer is received or input, and the expression level data is processed by processing software. The third machine learning model is constructed based on a machine learning algorithm of a generalized linear regression model, and the third machine learning model is adjusted and trained by the processed expression level data to obtain the trained third machine learning model.

The drug having the treatment effect refers to a drug that effectively eliminates or delays the progression of the patient with osimertinib-resistant lung cancer.

In some embodiments, the third machine learning model may generate a sensitive prediction probability and an insensitive prediction probability during operation and determine, based on whether the expression level data satisfies a judgment condition, whether the drug has treatment effects on the patient with osimertinib-resistant lung cancer. The sensitive prediction probability indicates the probability that the osimertinib treatment is effective to the patient. The insensitive prediction probability indicates the probability that the osimertinib treatment is ineffective to the patient.

The judgment condition includes that the sensitive prediction probability is greater than or equal to the insensitive prediction probability.

In some embodiments, when the expression level data satisfies the judgment condition, the output result is “The drug has a treatment effect on the patient with osimertinib-resistant lung cancer”. When the expression level data does not satisfy the judgment condition, the output result is “The drug has no treatment effect on the patient with osimertinib-resistant lung cancer”.

In one embodiment, gene expressions in NCI-H1975 and NCI-H1975/OR cells (Osim) were detected and analyzed by MeRIP-seq and RNA-seq. The NCI-H1975/OR cells (Osim) were osimertinib-resistant cells treated with osimertinib. The specific process includes comparing reads obtained from sequencing of the IP RNA library and input library (also referred to as the total RNA (input)) to a reference genome, and the abundance and probability of reads falling on the reference genome were higher for the IP RNA library compared to the input library. The reads of the IP RNA library landing on the reference genome formed a distinct reads-enriched region, which is referred to as a peak. Differential Peak analysis was performed using exompeak software, etc., and annotated with ANNOVAR software, etc. Reference genes may be standard genome sequences, which may be obtained from a third-party database (e.g., NCBI, etc.).

As shown in FIG. 8, the expression level of UQCC2 in NCI-H1975/OR cells (Osim) is higher than the expression level of UQCC2 in parental NCI-H1975 cells.

In a specific embodiment, the protein expression of UQCC2 in the NCI-H1975/OR cells (treated with Ctrl, Osim, or Lomi alone) was determined by western blot assay. The western blot assay was performed using the following antibody: anti-UQCC2 (antibody dilution ratio of 1:1000, Proteintech, #25781-1-AP). Anti-GAPDH (antibody dilution ratio of 1:50,000, Proteintech, #60004-1-Ig) was used as a loading control. Significance was calculated using GraphPad Prism software, etc.

As shown in FIG. 9, the column corresponding to Lomi is lower than those corresponding to Osim and Ctrl, demonstrating that the protein expression of UQCC2 is significantly reduced in NCI-H1975/OR cells (osimertinib-resistant) after lomitapide (Lomi) treatment.

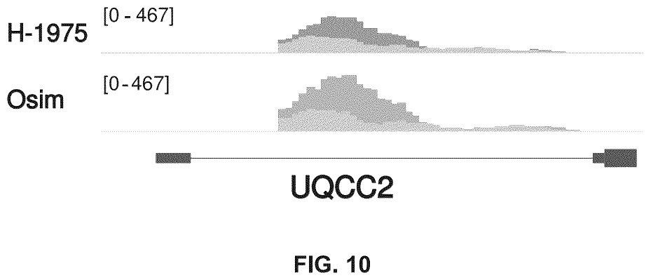

In one embodiment, after selecting a target gene of interest from difference peak results, the m⁶A methylation modification on the target gene was visualized and analyzed by IGV software, etc. FIG. 10 is a graph illustrating alteration results of m⁶A abundance of UQCC2 in non-small cell lung cancer parental cells (H1975) and in osimertinib-resistant cells (Osim) treated with osimertinib through IGV analysis.

As shown in FIG. 10, m⁶A abundance of UQCC2 is altered (higher peak and larger area) in H1975/OR cells (Osim) treated with osimertinib compared to parental cells (H1975) of non-small cell lung cancer. The UQCC2 m⁶A modification predominantly alters the intronic region during the induction of osimertinib resistance.

In some embodiments, the processor may determine indicator sequences corresponding to a plurality of future time points through a dynamic prediction model based on the expression level data, a drug parameter, and genomic data, and determine, based on the indicator sequences, whether the drug has the treatment effect on the patient with osimertinib-resistant lung cancer.

The drug parameter refers to information about the drug (e.g., lomitapide, osimertinib, etc.) being used by the patient with osimertinib-resistant lung cancer. In some embodiments, the drug parameter may include a type of drug (e.g., a small molecule, an antibody, etc.), a dosage, an administration frequency, a molecular structure, a drug target, a known mechanism, etc. The drug parameter may be obtained from the storage or manual input.

The genomic data refers to data related to genetic mutation sites in the patient with osimertinib-resistant lung cancer. In some embodiments, the genomic data may include information on genetic mutation sites related to lung cancer treatment and drug resistance, e.g., mutation status of 19del, L858R, T790M, C797S, etc., of EGFR gene.

In some embodiments, the genomic data may be obtained by a preset manner, for example, gene sequencing.

The indicator sequences refer to sequences composed of indicator values at the plurality of future time points. The indicator value may be data used to characterize the condition of the patient.

In some embodiments, the indicator value may include an autophagy score and other indicators. The indicator sequences may include an autophagy score sequence and other indicator sequences. The plurality of future time points may be pre-set based on historical experience, e.g., 2 hours, 12 hours, 24 hours, etc., after the patient takes the drug.

The autophagy score refers to a ratio of the expression level of the LC3 protein to the expression level of the p62 protein during treatment. An increase in the autophagy score at the plurality of future time points indicates that more autophagosomes are generated and more p62 proteins are being degraded by the autophagosomes, reflecting an increase in the activity of the autophagosomes and a possible improvement in the disease. A decrease in the autophagy scores at the plurality of future time points indicates that the autophagosomes are underactive or blocked, and the disease may worsen.

Other indicators may be indicators other than the autophagy score. In some embodiments, other indicators may include at least one of a tumor volume, a lesion diameter, a drug-resistant score, etc.

In some embodiments, the dynamic prediction model may be a fourth machine learning model. For example, the dynamic prediction model may include any one or a combination of a recurrent neural network (RNN) model, other customized model structures, etc.

In some embodiments, the dynamic prediction model may be obtained by training an initial dynamic prediction model with a plurality of sets of prediction training samples with prediction labels. The prediction training samples may include sample expression level data, a sample drug parameter, and sample genomic data. The prediction labels may be actual indicator sequences corresponding to the prediction training samples.

In some embodiments, the processor may obtain the prediction training samples and the prediction labels from the historical data. For example, the processor may obtain, from the historical data, recorded data of a plurality of patients receiving a plurality of drug treatments at first historical time, determine a plurality of preferred treatment cases from the recorded data, determine historical expression level data, a historical drug parameter, and historical genomic data corresponding to each preferred treatment case as the prediction training samples. The processor determines historical indicator sequences at a plurality of second historical times for each preferred treatment case as a prediction label of the corresponding prediction training sample. The historical indicator sequences corresponding to the preferred treatment case may be obtained by manual labeling. The first historical time is earlier than the plurality of second historical times.

The preferred treatment case refers to a case in the historical data where the patient has demonstrated significant improvement and stable control of the condition after taking the drug, and the indicator sequences have been recorded.

In some embodiments, the significant improvement and stable control of the condition may be demonstrated as follows. The final tumor volume or lesion diameter of the patient after treatment shows a reduction exceeding a volume reduction threshold or a diameter reduction threshold compared to that before the treatment. The final drug-resistant score of the patient shows a decline surpassing a drug-resistant score decrease threshold, and the autophagy score exhibits an increase exceeding an autophagy score increase threshold compared to that before the treatment.

In some embodiments, the volume reduction threshold, the diameter reduction threshold, the drug-resistant score decrease threshold, and the autophagy score increase threshold may be preset empirically.

The training process of the dynamic prediction model is similar to that of the molecular generation model, and more descriptions regarding the training process of the dynamic prediction model may be found in the related descriptions of training the molecular generation model.

In some embodiments, the processor may determine, based on the indicator sequences, whether the drug has the treatment effect on the patient with osimertinib-resistant lung cancer. For example, the processor may determine whether the drug has the treatment effect on the patient with osimertinib-resistant lung cancer based on whether the indicator sequences trigger a progressive disease (PD) event.

The PD event may reflect the progression of a deterioration in the patient's condition. In some embodiments, the PD event may be pre-set based on historical experience. For example, if the increase in tumor volume at any time point in the indicator sequences compared to the minimum value of tumor volume in the indicator sequences exceeds the volume expansion threshold, the PD event is determined to have occurred. As another example, if the increase in lesion diameter at any time point in the indicator sequences compared to the minimum value of lesion diameter in the indicator sequences exceeds a diameter expansion threshold, the PD event is determined to have occurred. As yet another example, if the increase in the drug-resistant score at any time point in the indicator sequences compared to the minimum value of the drug-resistant score in the indicator sequences exceeds a drug-resistant score increase threshold, the PD event is determined to have occurred.

As yet another example, if the decrease in the autophagy score at a single time point in the indicator sequences compared to the highest value of the autophagy score in the indicator sequences exceeds an autophagy score decrease threshold, the PD event is determined to have occurred.

The volume expansion threshold, the diameter expansion threshold, the drug-resistant score increase threshold, and the autophagy score decrease threshold may be empirically preset.

In some embodiments, the processor determines that the drug has the treatment effect on the patient with osimertinib-resistant lung cancer if the indicator sequences do not trigger the PD event. The processor determines that the drug has no treatment effect on the patient with osimertinib-resistant lung cancer if the indicator sequences trigger the PD event.

According to some embodiments of the present disclosure, by introducing the dynamic prediction model and combining the expression level data, the drug parameter, and the genomic data of the UQCC2 protein, multimodal and time-series prediction of the treatment effect on the patient with osimertinib-resistant lung cancer is achieved. Furthermore, according to the indicator sequences such as the autophagy score sequence predicted by the dynamic prediction model, the disease progression may be preemptively monitored and whether the drug has the treatment effect on the patient with osimertinib-resistant lung cancer may be determined at a molecular level, thereby improving the scientific rigor and personalization of determining whether the drug has the treatment effect on the patient.

In some embodiments, the processor constructs a query vector based on the expression level data, clinical information data, and the drug parameter, performs a vector query in a vector database, obtains a plurality of reference vectors that satisfy a matching condition with the query vector, and determines a target dynamic threshold based on a plurality of reference thresholds corresponding to the plurality of reference vectors. The processor constructs a target change curve corresponding to the indicator sequences and determines a target curve area enclosed by the target change curve and a target baseline, and determines, based on a comparison result of the target curve area with the target dynamic threshold, whether the drug has a treatment effect on the patient with osimertinib-resistant lung cancer.

The clinical information data refers to patient-related data. In some embodiments, the clinical information data may include the age, the gender, the tumor stage, the smoking history, and the mutation site of the EGFR gene of the patient. The clinical information data may be obtained by input from a healthcare provider.

In some embodiments, the processor may construct a feature vector based on the expression level data, the clinical information data, and the drug parameter and determine the feature vector as the query vector.

In some embodiments, the processor may select, from the historical data, the historical expression level data, historical clinical information data, a plurality of historical treatment records of different patients, and the historical drug parameter, construct a historical feature vector (denoted as a reference vector) based on the historical expression level data, the historical clinical information data, and the historical drug parameter corresponding to each patient, compute a reference threshold corresponding to each reference vector, and obtain the vector database.

The reference threshold refers to a dynamic threshold corresponding to the reference vector. The dynamic threshold refers to a critical value used to determine whether the drug has the treatment effect. In some embodiments, the dynamic threshold includes a positive threshold and a negative threshold. The positive threshold indicates the lower boundary with the treatment effect. The negative threshold indicates the upper boundary without treatment effect.

In some embodiments, for each reference vector, the processor may construct an autophagy score curve based on the autophagy score sequence corresponding to the reference vector, and determine the reference threshold corresponding to the reference vector based on the autophagy score curve. The autophagy score curve is a smooth curve, the horizontal coordinate is the plurality of future time points, and the vertical coordinate is the autophagy score.

In some embodiments, the processor may determine a curve area enclosed by the autophagy score curve and a baseline, and determine the reference threshold based on the curve area and an actual treatment result (which may be determined by determining whether the PD event occurs in the historical indicator sequence) of the drug on the patient.

The curve area includes a positive area and a negative area. The positive area is an area enclosed by the baseline and the portion of the autophagy score curve above the baseline. The negative area is an area enclosed by the baseline and the portion of the autophagy score curve below the baseline.

For example, if the drug has a treatment effect on the patient (no PD event occurs), the positive area of the curve area is determined as the positive threshold of the reference threshold. If the drug has no treatment effect on the patient (the PD event occurs), the negative area of the curve area is determined as the negative threshold of the reference threshold.

In some embodiments, the processor takes the historical autophagy score corresponding to a first time point in the historical indicator sequence as the baseline value, and plots a straight line parallel to the horizontal axis as the baseline.

In some embodiments, the matching condition may be that the query vector has a similarity to the reference vector that is greater than a similarity threshold. The similarity of vector is negatively correlated to a vector distance, and the vector distance includes Euclidean distance, etc. The similarity threshold is pre-set based on historical experience.

The target dynamic threshold refers to a dynamic threshold used to determine whether the drug has the treatment effect on the patient with osimertinib-resistant lung cancer.

In some embodiments, the processor may determine the target dynamic threshold based on the plurality of reference thresholds corresponding to the plurality of reference vectors. For example, the processor may determine an average of negative thresholds in the plurality of reference thresholds as a negative threshold of the target dynamic threshold, and determine an average of positive thresholds in the plurality of reference thresholds as a positive threshold of the target dynamic threshold.

The target change curve, the target baseline, and the target curve area are the autophagy score curve, the baseline, and the curve area constructed based on the predicted indicator sequence. The target curve area includes the positive area and the negative area. The target change curve, the target baseline, and the target curve area are constructed in a manner similar to the autophagy score curve, the baseline, and the curve area. More descriptions regarding the manner for constructing the target change curve, the target baseline, and the target curve area may be found in the above descriptions related to the autophagy score curve, the baseline, and the curve area.

In some embodiments, the processor may determine whether the drug has the treatment effect on the patient with osimertinib-resistant lung cancer based on the comparison result of the target curve area to the target dynamic threshold. For example, if the data corresponding to a preset count of future time points in the target change curve are located below the target baseline, and the negative area in the target curve area is smaller than the negative threshold in the target dynamic threshold, it indicates that the negative area is large and the disease continues to deteriorate, indicating that the drug has no treatment effect on the patient with osimertinib-resistant lung cancer. The preset count is pre-set based on historical experience.

As another example, if the data corresponding to the preset count of future time points in the target change curve are above the target baseline, and the positive area in the target curve area is greater than the positive threshold in the target dynamic threshold, it indicates that the disease has been in remission or control for a long time and that the drug has a treatment effect on the patient with osimertinib-resistant lung cancer.

As yet another example, if the target curve area is located between the positive threshold and the negative threshold of the target dynamic threshold, it indicates that it is unclear whether the drug has a treatment effect on the patient with osimertinib-resistant lung cancer, and further observation is required.

According to some embodiments of the present disclosure, by introducing the personalized dynamic threshold generated by multi-source information (e.g., the expression level data of UQCC2 protein, the clinical information data, and the drug parameter, etc.), it is possible to more accurately match the disease evolution characteristics of different patients, improving the sensitivity and reliability of judging whether the drug has the treatment effect on the patient with osimertinib-resistant lung cancer, and significantly enhancing the adaptability and practical value of the drug generation model in real clinical scenarios.

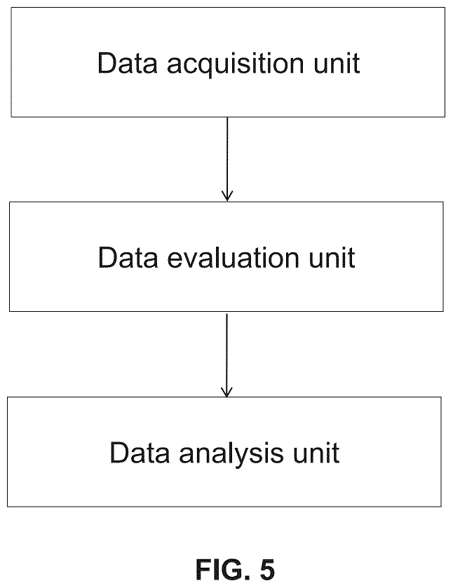

FIG. 5 is a schematic diagram illustrating an exemplary computer-assisted system for predicting a drug treatment effect on a patient with osimertinib-resistant lung cancer according to some embodiments of the present disclosure. Specifically, the computer-assisted system includes a data acquisition unit, a data evaluation unit, and a data analysis unit.

The data acquisition unit is configured to obtain the expression level data of UQCC2 in the patient with osimertinib-resistant lung cancer after treatment with a drug.

The data evaluation unit includes stored reference data and a data processor, and the data processor includes an algorithm for comparing the expression level data of UQCC2 with the stored reference data. The data processor is configured to execute algorithms, etc., and includes a processor, etc., as shown in FIG. 3.

The data analysis unit is configured to determine whether the drug has a treatment effect on the patient with osimertinib-resistant lung cancer based on the expression level data of UQCC2 in the patient with osimertinib-resistant lung cancer after treatment with a drug and the stored reference data.

An embodiment of the present disclosure provides a computer-assisted device for predicting a drug treatment effect on a patient with osimertinib-resistant lung cancer, the device includes a storage and a processor, and the storage is configured to store program instructions. The processor is configured to invoke the program instructions and realize the computer-assisted method for predicting the drug treatment effect on the patient with osimertinib-resistant lung cancer when the program instructions are executed.

In some embodiments, the processor may include a central processing unit (CPU), an application-specific integrated circuit (ASIC), an application-specific instruction set processor (ASIP), a graphics processing unit (GPU), a physics processing unit (PPU), etc., or any combination thereof. The storage may include a high-capacity storage, etc., or any combination thereof.

In some embodiments, the storage may be integrated in the processor. The data acquisition unit, the data evaluation unit, and the data analysis unit may be integrated into the processor.

An embodiment of the present disclosure also provides a non-transitory computer-readable storage medium storing a computer program. The computer-assisted method for predicting the drug treatment effect on the patient with osimertinib-resistant lung cancer is implemented when the computer program is executed by a processor.

FIG. 6 is a flowchart illustrating an exemplary UQCC2 and METTL3-based computer-assisted method for screening drugs according to some embodiments of the present disclosure. The UQCC2 and METTL3-based computer-assisted method includes the following operations.

In S601, UQCC2 protein data and METTL3 protein data are obtained.

In some embodiments, both the UQCC2 protein data and the METTL3 protein data include molecular structures, binding sites, etc. The UQCC2 protein data and the METTL3 protein data may be obtained from the storage or a third party database.

In one embodiment, the correlation between METTL3 and UQCC2 expression was investigated by western blot assay after knockdown of the gene expression of METTL3 or UQCC2 in H1975/OR cells through small interfering RNA (siRNA), etc. The knockdown may be a partial reduction in the expression level of a specific gene.

siMETTL3 (5′-3′) (siRNA for METTL3 gene knockdown): sense strand GCACUUGGAUCUACGGAAU(dT)(dT); antisense strand AUUCCGUAGAUCC AAGUGC(dT)(dT); siUQCC2 (5′-3′) (siRNA for UQCC2 gene knockdown): sense strand: GACUCCAUUCAAACUACUATT, antisense strand: UAGUAGUUUUGAAUGGAGUCTT.

In some embodiments, the western blot assay was performed using the following antibodies: anti-METTL 3 (antibody dilution ratio of 1:2000; #15073-1-AP; Proteintech) and anti-UQCC2 (antibody dilution ratio of 1:1000, Proteintech, #25781-1-AP). Anti-GAPDH (antibody dilution ratio 1:50,000, Proteintech, #60004-1-Ig) was used as a loading control. Significance was calculated using GraphPad Prism software, etc.

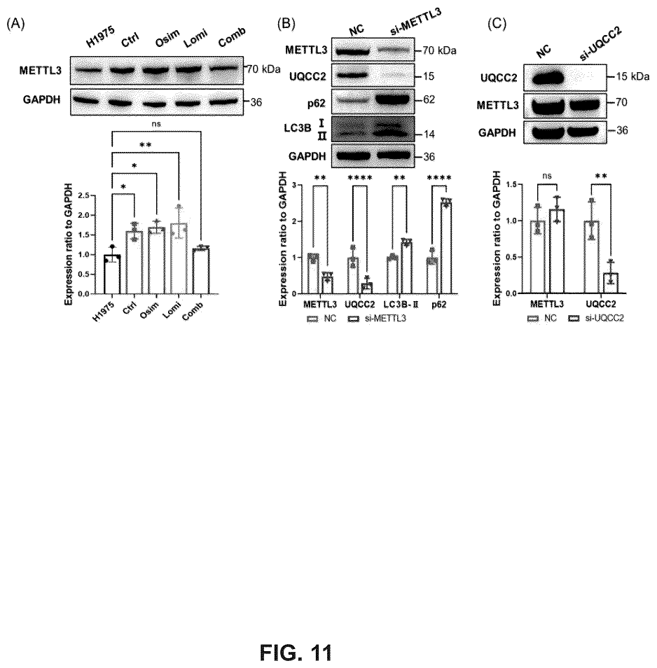

FIG. 11 is a graph illustrating expression levels of various biomarkers determined by western blot assay. FIG. 11A is a graph illustrating protein expression levels and corresponding statistical analysis of METTL3 in NCI-H1975 cells and NCI-H1975/OR cells (treated with Ctrl, Osim, or Lomi alone or in combination) as determined by western blot assay. FIG. 11B is a graph illustrating an effect and corresponding statistical analysis of METTL3 knockdown on protein expression levels of UQCC2, P62, and LC3. FIG. 11C is a graph illustrating an effect and corresponding statistical analysis of UQCC2 knockdown on protein expression levels of METTL3, P62, and LC3.

The experimental results, as shown in FIG. 11, demonstrate that METTL3knockdown downregulates the expression of UQCC2, whereas UQCC2 knockdown does not affect the expression of METTL3, which suggests that UQCC2 may be a novel downstream target of METTL3.

In S602, the binding site of the UQCC2 protein to the METTL3 protein is determined based on the UQCC2 protein data and the METTL3 protein data.

In one embodiment, the binding site is a specific site in a molecule that can form a stable interaction with a ligand. The binding site of proteins is usually formed by some amino acid residues that are separated from each other on a polypeptide chain and are spatially clustered together by folding of the peptide chain to form a specific spatial arrangement.

In S603, a candidate drug that simultaneously targets the binding site of the UQCC2 protein to the METTL3 protein is obtained through computer-assisted screening.

In one embodiment, the computer-assisted drug screening is a technique for drug screening utilizing a computer-assisted drug design manner. It can help researchers to quickly screen the candidate drug with strong binding ability to target proteins and potential drug efficacy among a plurality of small molecule compounds.

In one embodiment, the molecular docking is a manner of drug design based on characteristics of the receptor and the manner in which the receptor and the drug molecule interact. It is a theoretical simulation manner that focuses on the study of intermolecular (e.g., the ligand and the receptor) interactions and predicts their binding modes and affinities. This manner is widely used in the initial stages of drug development to help researchers quickly screen compounds with potential medicinal effects.

The molecular docking focuses on spatial matching and energy matching. The spatial matching refers to geometric complementarity between drug molecules and receptor proteins, while the energy matching refers to the minimization of the interactions between drug molecules and receptor proteins. For the calculation of geometric matching, manners such as grid-based Calculation or fragment growing, etc., are commonly used, while energy calculation uses manners such as simulated annealing or genetic algorithms, etc. Based on the degree and manner of simplification, the molecular docking may be categorized as rigid docking, semi-flexible docking, flexible docking, etc. The rigid docking does not change the conformation of the molecules involved in the docking during the calculation process but only changes the spatial position and orientation of molecules. The semi-flexible docking allows for a partial change in the conformation during the calculation. The flexible docking allows for more changes in the conformation.

In some embodiments, the molecular library used in virtual screening are mainly ZINC, PubChem, DrugBank, ChEMBL, ChemDB, HMDB, BindingDB, SMPDB, etc. In addition, several commercial databases such as ChemDiv, Enamine, Lifechemicals, Specs, Chembridge, Maybridge, Microsource, Vitas-M, Interbioscreen, etc., are also often used for virtual screening.

In one embodiment, the process of computer-assisted screening is as follows.

The molecular docking of the screened small molecule compounds with the METTL3 protein is performed to calculate the affinity or binding energy of the targeted receptor to obtain scores, and the candidate drug is ranked according to the scores. The affinity may indicate the strength of the propensity of the small molecule compounds to bind to the METTL3 protein. The binding energy may indicate the change in free energy released when the small molecule compounds bind to the METTL3 protein.

In some embodiments, the scores are ranked, and the top n small molecule compounds are selected to obtain a candidate compound (i.e., a candidate drug), n being a natural number greater than or equal to 1.

In one embodiment, the process of computer-assisted screening includes obtaining the molecular structures of the UQCC2 protein and the METTL3 protein, transferring the molecular structures to a library of pharmacophore modules for matching, clustering all the action sites based on the interaction patterns of UQCC2 and METTL3 to obtain a pharmacophore module, and obtaining the candidate drug by transferring the pharmacophore module to a database of molecular compounds to perform a high-throughput screening.

In one embodiment, the process of computer-assisted screening also includes first obtaining the molecular structure of a small molecule inhibitor that inhibits METTL3 and/or the molecular structure of a small molecule inhibitor that inhibits UQCC2, and then screening the molecular structure of the small molecule inhibitor to obtain a small molecular library with a similar structure, then performing the molecular docking on the small molecular library with a similar structure and the binding site to obtain scores of docking molecules, and finally ranking the scores to obtain the candidate drug.

In one embodiment, the process of computer-assisted screening further includes obtaining the candidate drug by screening the protein analog, the antibody, or the RNA drug by the binding site.

In one embodiment, the process and manner of designing a drug, such as the protein analog or antibody, based on the protein may be summarized as target protein determination, protein structure analysis, determination of an interaction between the drug and the protein, drug design, and drug synthesis and validation.

The target protein determination includes first determining the target protein, i.e., the target, for which the drug is to be designed. The target may be a known disease-associated protein, a viral antigen, other biomolecules, etc.

The protein structure analysis includes performing structural analysis of target proteins to understand features, e.g., a three-dimensional conformation, a surface conformation, and a subdomain structure, etc., of the target protein through X-ray crystal diffraction, nuclear magnetic resonance, etc.

The determination of the interaction between the drug and the protein includes studying the mechanism of the interaction between the drug and the target protein, including binding sites, binding modes, and binding kinetics, etc., through computer simulation and laboratory experiments.

The drug design includes designing a drug molecule that can bind specifically to the target protein according to the mechanism of the interaction between the drug and the target protein, including choosing a suitable drug type, designing the chemical structure of the molecule, optimizing the pharmacodynamic and pharmacokinetic properties of the molecule, etc.

The drug synthesis and validation includes preparing the drug molecule through chemical synthesis, etc., and validating the drug molecule for bioactivity, safety, and pharmacokinetic properties, etc., including different phases of cellular experiments, animal experiments, and clinical trials.

In one embodiment, the protein analog refers to a substance that has a structure and function similar to a protein in the body. The protein analog may mimic the function of proteins and thus play a vital role in disease treatment. The antibody refers to immunoglobulin produced by plasma cells differentiated from B-lymphocytes by the body's immune system in response to antigenic stimulation, which may specifically bind to a corresponding antigen. The structure of the antibody is mainly divided into a constant region and a variable region. The RNA drug refers to a class of drugs designed based on the properties of RNA and the action mechanism in the cell. RNA may be used in cells as messenger RNA (mRNA) to direct protein synthesis and as microRNA (miRNA) to regulate gene expression. Rationally designed RNA drugs may therefore be used to regulate the gene expressions within cells to treat diseases.

In one embodiment, the small molecule compounds with inhibitory effects are screened by mixing the small molecule compounds with a UQCC2 protein solution and then calculating the inhibition rate.

In one embodiment, the general operations for performing an inhibitory activity assay for a small molecule compound include suitable target protein selection, small molecule compound preparation, an enzyme activity assay, an experimental operation, data analysis, and result interpretation and discussion.

The suitable target protein selection includes selecting a suitable target protein as an experimental subject according to the research purpose and the disease target. The target protein is ensured to have potential interactions with the small molecule compounds under study.

The small molecule compound preparation includes synthesizing or purchasing desired small molecule compounds and ensuring their purity and structural accuracy. As needed, the small molecule compounds may be modified or adapted to optimize their inhibitory activity.

The enzyme activity assay includes designing an appropriate enzymatic activity assay manner to evaluate the inhibitory activity of the small molecule compounds against the target protein, which may be achieved through techniques such as fluorescence resonance energy transfer (FRET), radioisotope labeling, and an enzyme-linked immunosorbent assay (ELISA), etc. The manner of the assay is ensured to be reliable, sensitive, and accurately reflects the interaction of the small molecule compounds with the target protein.

The experimental operation includes mixing different concentrations of the small molecule compounds with the target protein and incubating for a period of time under appropriate conditions, and then measuring the enzyme activity. Depending on the experimental design, a control group and an experimental group may be set up to compare the inhibitory activities of different small molecule compounds.

The data analysis includes determining the IC50 value (i.e., the concentration of the compound required to inhibit 50% of the enzyme activity) of the small molecule compounds by statistically analyzing the experimental data. The smaller the IC50 value, the stronger the inhibitory activity of the small molecule compounds. In addition, a dose-effect curve may be plotted to visualize the inhibitory activity of the small molecule compounds.

The result interpretation and discussion includes analyzing the inhibitory activity of the small molecule compounds against the target protein based on experimental results and discussing in conjunction with their structural characteristics and physicochemical properties. Possible binding modes, action mechanisms, and comparison with known inhibitors are explored. In addition, the effects of the small molecule compounds on cells or organisms may be further investigated to assess their potential as drug candidates.

In one embodiment, an inhibitory activity assay is performed on the candidate drug of the protein analog, the antibody, or the RNA drug, employing co-immunoprecipitation analysis for unbiased screening of cells co-transfected with V5-tagged protein analogs, antibodies, or RNA drugs, and Flag-STING.

In one embodiment, co-immunoprecipitation (Co-IP) is a classical approach used for studying protein-protein interactions based on the specific interaction between antibodies and antigens and serves as an effective approach for determining physiological interactions between two proteins within intact cells. The principle is to identify relevant protein-protein interactions in vivo by indirectly capturing a protein that binds to a specific target protein using a target protein-specific antibody. The specific antibody captures the target protein in the sample to form an antibody-target protein complex. The complex is then immobilized and precipitated using a bead-like support (e.g., Nanoab-Agarose, etc.) that can bind to the antibody, while proteins interacting with the target protein also precipitate. Finally, after washing away non-specific proteins that bind to the target protein, analysis by SDS-PAGE or detection by western blot, etc., is performed.

In one embodiment, co-transfection refers to the simultaneous transfection of two independent nucleic acid molecules, e.g., plasmid DNA and siRNA, and is a commonly used procedure for stabilizing transfection. The physical process of the co-transfection, in which two nucleic acid molecules are integrated into the same integrating sequence and expressed within the same transfected cell, involves the selection of correct co-transfection reagents and the use of advanced lipid nanoparticle technology to achieve superior transfection performance and reproducible results.

In one embodiment, the process of the computer-assisted drug screening technology includes one or more of protein-small molecule docking, protein-protein docking, and protein-nucleic acid docking.

In one embodiment, the protein-small molecule docking refers to a computational simulation process in which the structures of proteins and small molecules (e.g., drug molecules) are docked together by certain algorithms and procedures. This process may be used to study protein-small molecule interactions and possible biological functions. In protein-small molecule docking, software such as DOCK is usually used for computational simulation. DOCK is a highly automated drug-design software that enables docking between small molecule ligands and biomolecule receptors. It employs a fragment-based scoring methodology that enables fast and accurate docking. RosettaDock is commonly used software for protein-protein docking that employs a fragment-based scoring methodology to enable fast and accurate protein docking. This software enables precise adjustment of side chain conformations during docking and takes into account a variety of complex interactions such as hydrogen bonding, ionic bonding, and hydrophobic interactions.

In one embodiment, the protein-nucleic acid docking refers to a computational simulation process in which the structures of proteins and nucleic acids (e.g., DNA or RNA) are docked together by certain algorithms and programs. This process may be used to study the interaction between the proteins and nucleic acids, as well as the possible biological function. In protein-nucleic acid docking, software such as NAflex is usually used for computational simulation. NAflex is software developed specifically for predicting and designing nucleic acid structures, enabling accurate modeling and docking of DNA or RNA molecules. NAflex software employs a fragment-based scoring methodology that enables fast and accurate docking. It considers various complex interactions, e.g., hydrogen bonding, ionic bonding, hydrophobic interactions, etc., and can achieve precise adjustment of the side chain conformation during docking.

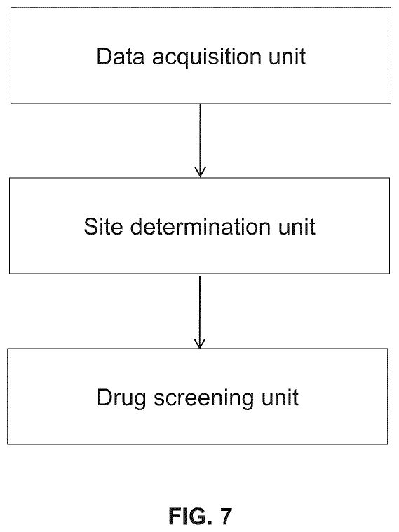

FIG. 7 is a schematic diagram illustrating an exemplary UQCC2 and METTL3-based computer-assisted system for screening drugs according to some embodiments of the present disclosure. The computer-assisted system includes a data acquisition unit, a site determination unit, and a drug screening unit.

The data acquisition unit is configured to obtain UQCC2 protein data and METTL3 protein data.

The site determination unit is configured to select the spatial structure of the UQCC2 protein and METTL3 protein complex and determine a binding site of the complex as the binding site of the target drug.

The drug screening unit is configured to adopt the computer-assisted method for screening drugs to obtain a candidate drug of a target binding site.

The present disclosure provides a UQCC2 and METTL3-based computer-assisted device for screening drugs, and the computer-assisted device includes a storage and a processor. The storage is configured to store program instructions. The processor is configured to invoke the program instructions and realize the UQCC2 and METTL3-based computer-assisted method for screening drugs as described above when the program instructions are executed.

The present disclosure also provides a non-transitory computer-readable storage medium having a computer program thereon, the computer program being executed by the processor to implement the UQCC2 and METTL3-based computer-assisted method for screening drugs as described above.

The present disclosure also provides a method for regulating autophagy, including administering a regulating reagent of UQCC2.

In one embodiment, the method for regulating autophagy includes regulating an autophagy-related marker. The autophagy-related marker includes autophagosome markers, autophagy-related genes and proteins, and lysosomal markers, etc.

In some embodiments, the autophagosome markers include, but are not limited to, LC3, p62, etc. The autophagy-related genes and proteins include, but are not limited to, Atg5, Atg12, Beclin1, or the like. The lysosomal markers include, but are not limited to, LAMP1, LAMP2, tissue proteases, etc. As an embodiment of the present disclosure, the autophagy-related marker for regulation is selected from LC3 and p62.

In some embodiments, the method for regulating autophagy includes increasing the expression level of the autophagy-related marker, and the manner for detecting the expression level of the autophagy-related marker includes western blot, immunofluorescence staining, RT-PCR, immunohistochemistry, ELISA, or the like.

In some embodiments, the regulating reagent includes an inhibitor of UQCC2. The inhibitor refers to any substance that inhibits the activity of the UQCC2 protein, the stabilization of the UQCC2 gene or protein, the expression level of UQCC2, the effective action duration of the UQCC2 protein, and the activity of UQCC2.

In one embodiment, the inhibitor includes a nucleic acid inhibitor, a protein inhibitor, a protein hydrolase, a protein binding molecule, and combinations thereof.

In one embodiment, the nucleic acid inhibitor includes an interfering molecule that targets a sequence of UQCC2 or a transcript thereof and can suppress gene expression or gene transcription of UQCC2, including, but not limited to, shRNA, siRNA, ribozyme, antisense oligonucleotide, dsRNA, microRNA, zinc finger, and CRISPR/Cas9, or constructs capable of expressing or forming shRNA, siRNA, ribozyme, antisense oligonucleotide, dsRNA, microRNA, zinc finger, and CRISPR/Cas9. The protein inhibitor includes substances capable of inhibiting the UQCC2 protein, etc. The protein hydrolase includes enzymes capable of catalyzing hydrolysis of UQCC2 proteins, etc. The protein binding molecule includes substances that bind specifically to the UQCC2 protein, e.g., antibodies or ligands capable of inhibiting the activity of the UQCC2 protein, etc.

In one embodiment, the siRNA may include partially purified RNA, highly purified RNA (OD 260/280 ratio being greater than 1.8), synthetic RNA, or recombinantly produced RNA, and modified RNA that is different from the natural RNA by the addition, deletion, substitution, and/or alteration of one or more nucleotides, etc. The alteration may include the addition of non-nucleotide substances, for example, the addition to the termini of the siRNA or to one or more internal nucleotides of the siRNA, or modifications that stabilize siRNA against nuclease digestion(e.g., the use of 2′-substituted ribonucleotides or modifications to the sugar-phosphate backbone), or the replacement of one or more of the nucleotides in the siRNA with deoxyribonucleotides, etc.

In one embodiment, the ribozyme is a class of RNA that may be engineered to enzymatically cleave and blunt other RNA targets in a specific sequence-dependent manner. The ribozyme and its delivery manners are well known in the field (Hendry et al, BMC Chem. Biol., 4(1): 1(2004 ); Grassi et al, Curr. Pharm. Biotechnol., 5(4): 369-386 (2004); Bagheri et al, Curr. Mol. Med., 4(5): 489-506 (2004); Kashani-Sabet M., Expert Opin. Biol. Ther., 4(11): 1749-1755 (2004)), each of which is incorporated herein by reference in its entirety. By cleaving the target RNA, the ribozyme inhibits translation and therefore prevents expression of the target gene. The ribozyme may be chemically synthesized in the laboratory using manners known in the art and structurally modified to increase stability and catalytic activity. Alternatively, the ribozyme gene may be introduced into the cell by the gene delivery mechanism known in the art.

In one embodiment, the antisense oligonucleotide (antisense nucleic acid sequence) may include a nucleotide sequence complementary to the sense nucleic acid encoding the protein (e.g., complementary to the coding strand of a double-stranded cDNA molecule or complementary to UQCC2 mRNA). The antisense oligonucleotide and its delivery manners are well known in the field (Goodchild, Curr. Opin. Mol. Ther., 6(2):120-128 (2004); Clawson et al, Gene Ther., 11(17):1331-1341 (2004)), which is incorporated herein by reference in its entirety. The antisense oligonucleotide may be complementary to the complete coding strand of the target sequence, or only partially complementary thereto. The length of the antisense oligonucleotide may be, for example, 7, 10, 15, 20, 25, 30, 35, 40, 45, 50, 55, 60, 65, 70, 75, 80 or more nucleotides.

In a specific embodiment, the impact of UQCC2 knockdown in H1975/OR cells on the expression of the autophagosome markers LC3 and p62 was investigated by western blot assay. siUQCC2 (5′-3′): sense strand: GACUCCAUUCAAACUACUATT; antisense strand: UAGUAGUUUUGAAUGGAGUCTT.

In some embodiments, the western blot assay was performed using the following antibodies: anti-UQCC 2 (antibody dilution ratio of 1:1000, Proteintech, #25781-1-AP), anti-LC3 (antibody dilution ratio of 1:2000; #81004-1-RR; Proteintech), and anti-p 62 (antibody dilution ratio of 1:5000; #80294-1-RR; Proteintech). Anti-GAPDH (Antibody dilution ratio 1:50,000, Proteintech, #60004-1-Ig) was used as a loading control. Significance was calculated using GraphPad Prism software, etc.

The experimental results, as shown in FIG. 11, demonstrated that the UQCC2 knockdown resulted in the upregulation of the autophagosome markers LC3 and p62, and further demonstrated that the UQCC2 knockdown significantly activated autophagy.

In one embodiment, the expression of the autophagosome marker LC3 was analyzed by the immunofluorescence assay after the UQCC2 knockdown in H1975/OR cells. siUQCC2 (5′-3′): sense strand: GACUCCAUUCAAACUACUATT; antisense strand: UAGUAGUUUUGAAUGGAGUCTT.

In some embodiments, the H 1975/OR cells may be fixed with 4% formaldehyde, permeabilized with 0.2% Triton X-100, and blocked with 5% bovine serum albumin. The cells were then incubated with the indicated antibodies at 4° C. overnight and stained with fluorescently coupled secondary antibodies and DAPI solution. The H1975/OR cells were photographed under a fluorescence microscope (Leica DM6B Thunder). Anti-LC3 (antibody dilution ratio of 1:100; antibody used was Proteintech, No. 81004-1-RR).

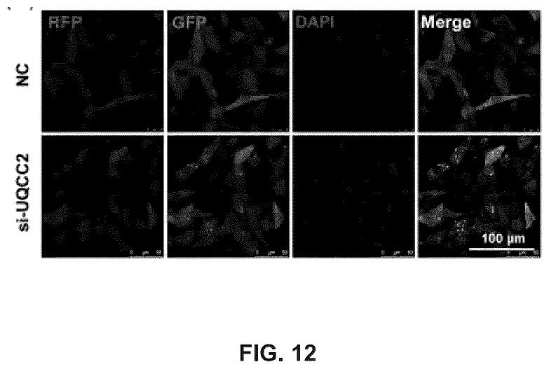

FIG. 12 is a graph illustrating the effect of UQCC2 knockdown on the expression of LC3 determined by an immunofluorescence assay.

The results of the assay, as shown in FIG. 12, demonstrated that the UQCC2 knockdown resulted in the up-regulation of the autophagosome marker LC3, and further demonstrated that the UQCC2 knockdown significantly activated autophagy.

Embodiments of the present disclosure provide any of the following manners.

First, a manner for detecting autophagy activation is provided. The manner determines the autophagy activation by detecting the UQCC2 level.

Second, a manner for diagnosing the osimertinib-resistant lung cancer is provided. The manner determines whether a subject has the osimertinib-resistant lung cancer by detecting the UQCC2 level in the subject.

In one embodiment, reagents for detecting the UQCC2 level include, for example, an oligonucleotide probe specifically recognizing the UQCC2 gene, a primer specifically amplifying the UQCC2 gene, or a binding agent of the protein specifically binding the protein encoded by the UQCC2 gene, etc.

In one embodiment, the probe refers to a molecule that binds to a specific sequence, or subsequence, or other portions of another molecule. Unless otherwise indicated, the term “probe” generally refers to a polynucleotide probe that binds to another polynucleotide (often referred to as a “target polynucleotide”) by complementary base pairing. Depending on the stringency of the hybridization conditions, the probe is capable of binding to a target polynucleotide that lacks full sequence complementarity with the probe. The probe may be directly or indirectly labeled. Hybridization manner includes, but is not limited to, solution-phase, solid-phase, mixed-phase, or in-situ hybridization assays, etc.

In one embodiment, the binding agent refers to all or part of a protein (protein, protein-like, or protein-containing) molecule capable of binding to the membrane protein using specific intermolecular interactions. The binding agent of the protein is, for example, a receptor for proteins, a lectin that binds proteins, an antibody against proteins, a peptidebody against proteins, a bispecific double binder or bispecific antibody form, etc. More specifically, the term “binding agent” refers to a polypeptide, and more specifically, a protein structural domain. A suitable protein domain is an element of the overall protein structure that is self-stabilizing and folds independently of the rest of the protein chain and is commonly referred to as a “binding domain”. The length of the binding domain varies from about 25 amino acids up to 500 amino acids and beyond. Many binding domains may be classified as folded and are recognizable, identifiable, 3-D structures. Some folds are so common across diverse proteins that they are given specific names.

The reagents for detecting the UQCC2 level provided in the present disclosure also include detectable markers. The detectable markers refer to compositions capable of generating detectable signals indicating the presence of a target polynucleotide in an assay sample. The detectable markers include, but are not limited to, radioisotopes, nucleotide chromophores, enzymes, substrates, fluorescent molecules, chemiluminescent moieties, magnetic particles, bioluminescent moieties, etc. Thus, detectable markers are any combinations capable of being detected by a device or manner. The device includes, but is not limited to, a spectroscopic, photochemical, biochemical, immunochemical, electrical, optical, or chemical detection device, or any other suitable device. In some embodiments, the detectable markers may be visually detectable without the aid of the device.

In one embodiment, the radioisotopes include, but are not limited to, 3H, 14C, 35S, 125I, 131I, etc. The enzymes include, but are not limited to, horseradish peroxidase, β-galactosidase, luciferase, alkaline phosphatase, acetylcholinesterase, etc. The fluorescent molecules include, but are not limited to, FITC, rhodamine, lanthanide phosphors, etc.

The reagents for detecting the UQCC2 level provided in this disclosure may be processed into various product formats.

In one embodiment, the product includes, but is not limited to, a chip, a kit, a test strip, a nucleic acid membrane strip, etc.

In one embodiment, the chip, also referred to as an array, refers to a solid support containing attached nucleic acid or peptide probes. The array typically includes a plurality of different nucleic acid or peptide probes attached to a substrate surface at specific known locations. The array, also referred to as “microarray”, may typically be fabricated using either a mechanical synthesis manner or a light-directed synthesis manner. The light-directed synthesis manner combines photolithography and solid-phase synthesis manners. The array may include flat surfaces, or may be nucleic acids or peptides on beads, gels, polymer surfaces, fibers such as optical fibers, glass, or any other suitable substrate. The array may be packaged in a manner that allows for diagnostic or other manipulation of a fully functional device.

In some embodiments, the nucleic acid membrane strip includes a substrate and an oligonucleotide probe immobilized on the substrate. The substrate may be any substrate suitable for immobilizing the oligonucleotide probe, e.g., a nylon membrane, a nitrocellulose membrane, a polypropylene membrane, a glass wafer, a silica wafer, a microminiature bead, etc.

The reagent kit of the present disclosure includes the reagent for detecting the UQCC2 gene or protein, which is selected from one or more of the following substances: a container, an instruction manual, a positive control, a negative control, a buffer, an auxiliary reagent, a solvent, a preservative, a protein stabilizer, etc. The components of the reagent kit may be packaged in an aqueous medium or in a lyophilized form. The container in the reagent kit typically includes at least one of a vial, a test tube, a long-necked vial, a Bowtie bottle, a syringe, or other containers, in which a single component may be placed, and which may be appropriately aliquoted. When more than one component is present in the reagent kit, the reagent kit typically also contains a second container, a third container, or other additional containers, with the additional components being placed segregated in a plurality of containers. However, different combinations of components may be contained in a single vial. The reagent kit of the present disclosure may seal a container containing reactants for commercial sale. The container may include, for example, an injection-molded or blow-molded plastic container, etc., in which the desired vial may be retained.

The reagent kit of the present disclosure includes, but is not limited to, a qPCR kit, an ELISA kit, an immunoblotting assay kit, an immunochromatography assay kit, an immunohistochemistry assay kit, a flow cytometry kit, an electrochemiluminescence assay kit, etc.

The present disclosure allows for the detection of the expression level of UQCC2 gene or protein using any known manner, including, but not limited to, RT-PCR, qRT-PCR, biochip assays, DNA blotting, in situ hybridization, immunoblotting, etc.

The various functional units in various embodiments of the present disclosure may be integrated in a single processing unit, or each unit may be physically present individually, or two or more units may be integrated in a single unit. The above integrated units may be implemented either in the form of hardware or in the form of software functional units.

The present disclosure also provides a method for screening drugs that activate autophagy in an osimertinib-resistant lung cancer. The method includes treating a culture system expressing or containing the UQCC2 gene or its encoded protein with a substance to be screened and detecting the expression or activity of the UQCC2 gene or its encoded protein in the system. When the substance to be screened inhibits the expression level or activity of the UQCC2 gene or the encoded protein, the substance to be screened is a candidate drug for activating autophagy.

In some embodiments of the present disclosure, the significant overexpression of UQCC2 in the osimertinib-resistant lung cancer is first discovered, and the functional interaction between UQCC2 and METTL3 is discovered, which provides an efficient and rapid method for diagnosing the patient with osimertinib-resistant lung cancer, predicting the drug treatment effect on the patient with osimertinib-resistant lung cancer, and screening drugs based on targeting UQCC2 and METTL3, demonstrating critical research significance for related disease prevention and treatment.

A person of ordinary skill in the art may understand that all or part of the operations in the various methods of the above embodiments may be accomplished by a program to instruct the relevant hardware, which may be stored in a non-transitory computer-readable storage medium. The non-transitory computer-readable storage medium includes a read-only memory (ROM), a random access memory (RAM), a diskette or CD-ROM, etc.

The above description of the embodiments is only for the purpose of understanding the method of the present disclosure and its core ideas. It should be noted that, for a person of ordinary skill in the art, several improvements and modifications may be made to the present disclosure without departing from the principles of the present disclosure, and these improvements and modifications will also fall within the scope of protection of the claims of the present disclosure.

1. A computer-assisted method for diagnosing a patient with osimertinib-resistant lung cancer, comprising:
receiving input data, wherein the input data includes expression level data of ubiquinol-cytochrome c reductase complex assembly factor 2 (UQCC2) in a patient with lung cancer; and
generating, based on the input data, an indication of whether the patient with lung cancer is the patient with osimertinib-resistant lung cancer through a machine learning model, wherein the lung cancer includes non-small cell lung cancer.

2. The computer-assisted method of claim 1, wherein the expression level data of UQCC2 includes a protein expression level and a nucleic acid expression level of UQCC2.

3. The computer-assisted method of claim 1, wherein a target drug is administered to the patient with lung cancer when the patient with lung cancer is the patient with osimertinib-resistant lung cancer, and the target drug includes lomitapide.

4. The computer-assisted method of claim 1, wherein a candidate drug is administered to the patient with lung cancer when the patient with lung cancer is diagnosed with osimertinib-resistant lung cancer, and a manner for obtaining the candidate drug includes:
determining a plurality of first candidate molecules and synthetic feasibilities of the plurality of first candidate molecules based on protein structural information, a chemical valence bonding rule, and a pharmacophore module through a drug generation model, the drug generation model being a machine learning model;
determining a second candidate molecule from the plurality of first candidate molecules based on the synthetic feasibilities; and
obtaining the candidate drug based on the second candidate molecule, wherein
the drug generation model includes a molecular generation model and an evaluation model, the molecular generation model is configured to determine the plurality of first candidate molecules based on the protein structural information, the chemical valence bonding rule, and the pharmacophore module, and the evaluation model is configured to determine the synthetic feasibilities of the plurality of first candidate molecules based on the plurality of first candidate molecules.

5. The computer-assisted method of claim 4, wherein the protein structural information includes information related to a spatial structure of a binding site of a UQCC2 protein to a methyltransferase 3, N6-adenosine-methyltransferase complex catalytic subunit (METTL3) protein.

6. The computer-assisted method of claim 4, wherein the obtaining the candidate drug based on the second candidate molecule includes:
obtaining a similar molecule through searching in a molecular library based on the second candidate molecule; and
determining the candidate drug based on the similar molecule.

7. The computer-assisted method of claim 4, wherein the drug generation model is obtained by a plurality of rounds of iterations, wherein
a process of one iteration of the plurality of rounds of iterations includes:
inputting training samples and random noise into an initial molecular generation model to obtain a type I molecule and setting a first label for the type I molecule, the training samples including sample protein structural information, a sample chemical valence bonding rule, and a sample pharmacophore module;
obtaining a type II molecule from a molecular library and setting a second label for the type II molecule;
updating parameters of an initial evaluation model based on the type I molecule, the type II molecule, the first label, and the second label;
keeping parameters of an updated initial evaluation model, changing the first label of the type I molecule to a third label and inputting the third label into the updated initial evaluation model, and updating, based on the third label and an output result of the updated initial evaluation model, parameters of the initial molecular generation model;
in response to determining that the updated initial evaluation model and an updated initial molecular generation model do not satisfy a preset convergence condition, designating the updated initial evaluation model and the updated initial molecular generation model as an initial evaluation model and an initial molecular generation model to be trained in a next round of iteration; and
in response to determining that the updated initial evaluation model and the updated initial molecular generation model satisfy the preset convergence condition, designating the updated initial evaluation model and the updated initial molecular generation model as the evaluation model and the molecular generation model.

8. A computer-assisted system for diagnosing a patient with osimertinib-resistant lung cancer, wherein the system executes the computer-assisted method for diagnosing the patient with osimertinib-resistant lung cancer of claim 1, comprising:
a data acquisition unit, configured to obtain the expression level data of UQCC2 in the patient with lung cancer;
a data evaluation unit, wherein the data evaluation unit includes stored reference data and a data processor, and the data processor includes an algorithm for comparing the expression level data of UQCC2 with the stored reference data; and
a drug-resistant identification unit, configured to determine whether the patient with lung cancer is the patient with osimertinib-resistant lung cancer based on the algorithm for comparing the expression level data of UQCC2 in the patient with lung cancer with the stored reference data, wherein
the lung cancer includes the non-small cell lung cancer.

9. A computer-assisted device for diagnosing a patient with osimertinib-resistant lung cancer, comprising a storage and a processor, wherein the storage is configured to store program instructions; the processor is configured to invoke the program instructions and realize the computer-assisted method for diagnosing the patient with osimertinib-resistant lung cancer of claim 1 when the program instructions are executed.

10. A non-transitory computer-readable storage medium storing a computer program, wherein the computer-assisted method for diagnosing the patient with osimertinib-resistant lung cancer of claim 1 is implemented when a processor executes the computer program.

11. A computer-assisted method for predicting a drug treatment effect on a patient with osimertinib-resistant lung cancer, comprising:
receiving input data, wherein the input data includes expression level data of UQCC2 in the patient with osimertinib-resistant lung cancer after treatment with a drug; and
generating, based on the input data, an indication of whether the drug has a treatment effect on the patient with osimertinib-resistant lung cancer through a machine learning model, wherein
the drug includes osimertinib, and the lung cancer includes non-small cell lung cancer.

12. The computer-assisted method of claim 11, further comprising:
determining indicator sequences corresponding to a plurality of future time points through a dynamic prediction model based on the expression level data, a drug parameter, and genomic data, wherein the indicator sequences include an autophagy score sequence and other indicator sequences, and the dynamic prediction model is a machine learning model; and
determining, based on the indicator sequences, whether the drug has the treatment effect on the patient with osimertinib-resistant lung cancer.

13. The computer-assisted method of claim 12, further comprising:
constructing a query vector based on the expression level data, clinical information data, and the drug parameter, performing a vector query in a vector database, and obtaining a plurality of reference vectors that satisfy a matching condition with the query vector;
determining a target dynamic threshold based on a plurality of reference thresholds corresponding to the plurality of reference vectors;
constructing a target change curve corresponding to the indicator sequences and determining a target curve area enclosed by the target change curve and a target baseline; and
determining, based on a comparison result of the target curve area with the target dynamic threshold, whether the drug has the treatment effect on the patient with osimertinib-resistant lung cancer.

14. The computer-assisted method of claim 11, wherein the expression level data of UQCC2 includes a protein expression level and a nucleic acid expression level of UQCC2.

15. A computer-assisted device for predicting a drug treatment effect on a patient with osimertinib-resistant lung cancer, comprising a storage and a processor, wherein the storage is configured to store program instructions; the processor is configured to invoke the program instructions and realize the computer-assisted method for predicting the drug treatment effect on the patient with osimertinib-resistant lung cancer of claim 11 when the program instructions are executed.

16. A non-transitory computer-readable storage medium storing a computer program, wherein the computer-assisted method for predicting the drug treatment effect on the patient with osimertinib-resistant lung cancer of claim 11 is implemented when a processor executes the computer program.
